(19) United States
(12) Patent Application Publication
(10) Pub. No.: US 2022/0233614 A1
(43) Pub. Date: Jul. 28, 2022

(54) GEL COMPOSITION COMPRISING VIABLE MICROORGANISMS

(71) Applicant: Lactobio A/S, Kobenhavn O (DK)

(72) Inventors: Charlotte Vedel, Copenhagen O (DK); Katja Billekop Olsen, Copenhagen O (DK); Soren Kjaerulff, Copenhagen (DK)

(21) Appl. No.: 17/617,567

(22) PCT Filed: Jun. 12, 2020
(86) PCT No.: PCT/EP2020/066311

§ 371 Date: Dec. 8, 2021

(30) Foreign Application Priority Data
Jun. 13, 2019 (DK) PA 2019 00718
Oct. 10, 2019 (DK) PA 2019 01196
Dec. 10, 2019 (DK) PA 2019 01451

(51) Int. Cl. A61K 35/747; C12N 1/20; A61K 9/06; A61K 47/44

(52) U.S. Cl. CPC A61K 35/747; C12N 1/20; C12R 2001/25; A61K 47/44; A61K 9/06

(57) ABSTRACT

A composition is provided that includes an oil gel having an oil, an oil-based viscosity increasing agent, and at least one viable microorganism.

### TECHNICAL FIELD OF THE INVENTION

The present invention relates to new gel composition comprising viable microorganisms. In particular the present invention relates to novel compositions for topical use on skin or mucous membranes comprising at least one viable probiotic microorganism.

### BACKGROUND OF THE INVENTION

There is considerable interest in the use of probiotic bacteria. Probiotics are live microorganisms that confer health benefits to the host when administered at adequate levels (FAO WHO, 2006). However, to exert these benefits, the microorganisms must remain viable during the processing and storage of the product containing live probiotics, considerable amount of research have been done to stabilize probiotics for oral consumption and ensure resistance to gastrointestinal fluids. Because probiotics are sensitive to a number of factors, including the presence of oxygen and acidic media, microencapsulation has been studied as a method of increasing the viability of probiotic cells. Microencapsulation of probiotics is a process that surrounds probiotic microorganisms in a polymeric membrane, protecting them and, in certain cases, allowing their release under specific conditions. The techniques commonly applied to encapsulate probiotics are extrusion, atomization or spray drying, emulsion, coacervation and immobilization in starch granules (Favaro-Trindade et al., 2011). Polysaccharides, such as alginate, gellan, K-carrageenan, and starch are the most commonly used materials in the microencapsulation of bifidobacteria and lactobacilli.

In order to meet the demand of skin care products comprising live microorganisms it is necessary to develop stable compositions for topical use which can maintain viability of the microorganisms as well as secure activation of the microorganism when applied on skin or mucous membranes.

Microencapsulation of microorganisms is well known in the art, however, these techniques are not developed for topical use and the microcapsules are designed to be dissolved in the intestinal tract releasing the microorganisms in the gut. When prior art microcapsules are applied on skin, the conditions on the skin will not dissolve the capsules and release the live microorganisms.

Topical formulations and products for pharmaceutical or cosmetic purposes are developed to have a long shelf life and to be stable towards contamination and spoilage caused by microorganisms. The stability of viable probiotics in these topical formulations are thus very limited, however, the use of probiotics in topical formulations could have a huge potential if viability can be maintained in the formulation. Topical formulations like creams, lotions, gels, mists inherently contain a high degree of water, i.e. in order to be suitably formulated into a gel, cream, foam, lotion, ointment etc. Evidently, the presence of such high degrees of water in these formulations, poses a problem for the storage of probiotics in their metabolically inactive condition. A second problem occurring in such topical formulations, is that these generally contain agents, which are not compatible with the survival of microorganisms; such as preservatives, surfactants, emulsifiers and other ingredients in order to protect such formulations against the growth of unwanted microorganisms as well as for forming stable emulsions. These agents, preservatives, will naturally be a major problem in the formulation of beneficial viable microorganisms. WO18002248 disclose a concept of formulating microorganisms in a 2-compartment system, protecting the microorganisms of the inner core compartment from the ingredients in the outer compartment once the content of both compartments is combined, this microencapsulation is for topical use, however, still this encapsulation comprises microcapsules of a size touchable to the skin and which needs to be rubbed into the skin to break the capsules. The capsules not broken by friction will then not release the viable microorganisms to the surface of the skin. Another problem will be the survival or activation on the skin when the capsules are broken and the viable probiotics released to the skin with the ingredients in the other compartment which can include preservatives inactivating the probiotic strain.

The use of viable probiotics for topical application is very limited and most products are based on lysates (inactivated dead bacteria) of the probiotic strain to overcome the problems of maintaining viability of the microorganisms in the topical composition. The problems observed when formulating live probiotic strains in gels, emulsions, lotions and the like for topical application on the skin of mammals are lack of viability and stability. Hence, it was an object of the present invention to provide a system allowing for long-term storage of viable microorganisms, which does not substantially harm such microorganisms upon use thereof and which does release the viable microorganisms when applied on the skin or on mucous membranes

It was surprisingly found that enrobing the microorganisms in an oil gel significantly stabilizes the viability of the microorganism, and a further surprising benefit was the ability to make an even distribution of the microorganism on the skin using the oil gel as compared to the oil.

It is an advantage of the present invention that embedding or mixing or dispersing or enrobing or coating the probiotic microorganisms in an oil gel enables long-term stability.

Hence, an improved stability would be advantageous, and in particular an increased viability would be advantageous.

Also advantageous is the improved distribution of the microorganisms in the oil gel, allowing the microorganisms to be spread more even on the skin surface or on mucous membranes.

Further advantage is the improved application of the oil gel as compared to the oil. It is an advantage of the present invention that the oil gel can be solidified allowing the viable microorganisms to be stable embedded in the oil gel and maintain viability in a solid or partly solid gel. Such stabilization of the gel structure reduces sedimentation of the microorganisms during storage. This is a new and advantageous formulation for viable microorganisms to be administered to mucous membranes as eg. the vagina.

### SUMMARY OF THE INVENTION

Thus, an object of the present invention relates to oil gel comprising viable microorganisms.

In particular the invention relates to an oil gel comprising an oil, a oil based viscosity increasing agent and a viable microorganism.

In particular, it is an object of the present invention to provide a gel that solves the above mentioned problems of the prior art with stability and viability of live microorganisms.

One aspect of the invention the oil based viscosity increasing agent is a hydrogenated oil.

Thus, one aspect of the invention relates to a composition comprising an oil, a polyurethane polymer and at least one viable microorganism.

The composition of the inventions comprises at least the following 3 components: oil, polyurethane polymer and a viable microorganism.

Another aspect of the present invention relates to a composition a polyurethane polymer which is based on vegetable oils. A further aspect of the invention the vegetable oil of the polymer is castor oil. More preferable, the polyurethane polymer comprises at least 10% w/w castor oil.

In another aspect of the invention the oil is selected from at least one of the following vegetable oils; jojoba oil, almond oil, sunflower seed oil, acai oil or almond sweet oil.

Yet another aspect of the present invention the composition is a gel.

Yet another aspect of the present invention the composition is an unclear gel.

Still another aspect of the present invention the viable microorganism is a lyophilized microorganism.

Another aspect of the invention the lyophilized microorganism is embedded in the oil in lumps.

In yet another aspect of the invention the lyophilized microorganisms are embedded in lumps with a diameter less than 120 μm.

And in still another aspect of the invention the composition is for treatment or prevention of a disorder or disease.

Another aspect of the invention is use of the composition as a prophylaxis medicament or medicament for treatment of a disease, dysfunction or disorder.

The present invention will now be described in more detail in the following.

### DETAILED DESCRIPTION OF THE INVENTION

### Definitions

Prior to discussing the present invention in further details, the following terms and conventions will first be defined:

By “embedding” or “mixing” or “dispersing” or “enrobing” or “coating” it is meant that the probiotic microorganism is dispersed within and fully enveloped by the oil gel. By “enveloped” it is meant to enclose or enfold completely within the oil. The oil gel is characterized by being solid or partly solid or liquid.

In a preferred embodiment the oil gel is solid or partly solid at temperatures below 37 degrees Celsius.

It is preferred that the probiotic culture products disclosed herein remain essentially dry, and that they contain no more than a trace of water. The use of substantial quantities of water in processing is typically incompatible with the coating oil and the product stability.

The oil with embedded viable microorganisms can be used for topical application directly as an oil composition.

The oil can be processed into a liquid oil gel, a partly solid oil gel or a solid oil gel wherein the microorganisms is embedded in a concentration from 0.01 to 95% of the composition.

The oil gel embedded viable probiotics can be further processed into an emulsion comprising a hydrophilic phase from 0.01 to 5% of the composition.

In one preferred embodiment of the invention the composition is an oil gel consisting of a hydrophobic oil phase wherein the hydrophobic phase comprises embedded micro-organisms.

In an embodiment of the present invention the oil gel may be an organogel or an oleogel.

Preferably, the oil gel comprises an organogelator.

In an embodiment of the present invention the oil based viscosity increasing agent may be an organogelator.

Structuring edible oil with an organogelator is used in the food industry for replacing trans fats without increasing the amount of saturated fats. An organogel, also called oleogel, is a class of gel made of a liquid organic phase immobilized by a three-dimensional network formed by an organogelator.

Although many types of organogelators have been developed, plant waxes and hydrogenated vegetable oils such as Rapeseed wax (hydrogenated rapeseed oil), candelilla wax (Euphorbia cerifera cera), rice bran wax (Oryza sativa Bran Cera), berry wax (Rhus verniciflua peel cera/Rhus succedanea fruit cera), Oliwax (hydrogenated olive oil), Tea wax (Camellia sinensis cera), Myrica fruit wax (Myrica cerifera fruit wax), sunflower wax (Hydrolyzed sunflower seed wax), Sunflower seed wax (Helianthus annuus Seed cera, ascorbyl palmitate, tocopherol), Castor wax (Hydrogenated castor oil), carnauba wax (Copernicia cerifera cera) or any other vegetable based wax (hydrogenated vegetable oil) are of great interest due to their availability, low cost, and great gelling ability. When used as organogelators the waxes can be mixed to create a gel with particular physical properties.

Organogelator is preferable used in concentrations from 0.1 to 40% (w/w) of the oil. More preferable the concentration of the organogelator is 0.5 to 20% and even more preferable the concentration is 1 to 17%.

Some plant waxes have demonstrated potential health benefits. For example, when rats were fed with diets containing up to 1% sunflower wax their serum cholesterol levels were lowered. It was also found that gelation of oil with an organogelator can control the release of lipids into the blood which, in turn, attenuates the post-prandial increases in triglycerides, free fatty acids, and insulin levels induced by the acute ingestion of fat. Therefore, one can expect multiple health benefits from food products that have been structured using organogels.

In addition, most plant waxes are by-products. For example, sunflower wax is produced during the refining of sunflower oil. Therefore, developing products containing wax-based organogels facilitates the use of these agricultural by-products.

Polymers and synthetic waxes can also be used to gel the oil. The polymer suited for the invention are hydrogenated oils and polyurethane polymers and co-polymers being able to gel oils. Examples of such polyurethane polymers are disclosed in WO18185432. Only few polyurethane polymers are able to gel oils and examples of these are Oilkemia 5S polymer from Lubrisol and EstoGel M polymer from Polymerexpert. These polyurethane polymers comprise caprylic/capric triglycerides (castor oil) and are typically co-polymers of castor oil and polyurethane. The polymer of the invention is a polyurethane based on vegetable oils. Some vegetable oils used for production of polyurethane may need chemical modifications before polymerization.

In a preferred embodiment of the invention the polyurethane polymer is based on Castor oil.

In a preferred embodiment the polymer comprises more than 10% w/w castor oil, in a more preferred embodiment the polyurethane polymer comprises more than 20% w/w castor oil.

The invention is not limited to these two commercially available polyurethane products but to any polyurethane polymer/co-polymer product being able to gel oils.

The caprylic/capric triglyceride and polyurethane polymers are used in the oil in a concentration from 0.1% (w/w) to 20% (w/w). Preferable in the concentration from 0.3% (w/w) to 10% (w/w) and more preferable from 0.5% (w/w) to 6% (w/w).

Synthetic waxes include microcrystalline wax which is produced by de-oiling petrolatum as part of its refining process. Paraffin wax is also derived from petroleum. Ozokerite, ceresin, and montana waxes are originally mineral waxes which are derived from coal and shale.

Ozokerite for cosmetics are nowadays synthesized from petroleum, exactly like microcrystalline waxes. Ozokerites reduce the brittleness of stick preparations and add strength (hardness) and stability to the gel.

Emulsifiers can be used to stabilize the composition, emulsifiers for topical emulsions are known in the art and can be selected from fractionated lecithins enriched in either phosphatidyl choline or phosphatidyl ethanolamine, or both; mono and diglycerides thereof; monosodium phosphate derivatives of mono and diglycerides of edible fats or oils; lactylated fatty acid esters of glycerol and propylene glycol; hydroxylated lecithins; polyglycerol esters of fatty acids; propylene glycol; mono and diester of fats and fatty acids; DATEM (diacetyl tartaric acid esters of mono and diglycerides); PGPR (polyglycerol polyricinoleate); polysorbate 20, 40, 60, 65 and 80; sorbitan monostearate; sorbitan tristearate, oat extract; and the like. The emulsifier is not limited by this list.

In a preferred embodiment of the invention the oil gel does not comprise any emulsifiers.

In a further preferred embodiment of the present invention the oil gel composition provides an anoxic environment around the microorganism. In an embodiment of the present invention the oil gel composition may be an anoxic composition.

In a preferred embodiment of the present invention the oil gel composition does not comprise a preservative.

In a further preferred embodiment of the present invention the oil gel composition does not comprise a surfactant.

Preferably the oil gel composition of the present invention does not comprise a preservative, and a surfactant; or a preservative, a surfactant, and an emulsifier; or a preservative, and an emulsifier; or a surfactant, and an emulsifier.

The present invention relates to live microorganisms including any bacteria, archaea, phages, viruses, yeast or fungi or any combinations thereof.

Examples of suitable probiotic microorganisms include yeasts such as Saccharomyces, Debaromyces, Candida, Pichia and Torulopsis, moulds such as Aspergillus, Rhizopus, Mucor, and Penicillium and Torulopsis and bacteria such as the genera Bifidobacterium, Bacteroides, Clostridium, Fusobacterium, Melissococcus, Propionibacterium, Streptococcus, Enterococcus, Lactococcus, Staphylococcus, Peptostrepococcus, Bacillus, Pediococcus, Micrococcus, Leuconostoc, Weissella, Aerococcus, Oenococcus, Cutibacterium and Lactobacillus.

The most commonly used probiotics are strains of the lactic acid bacteria (LAB). These are considered non-pathogenic and are used as probiotic bacteria in general to improve gastrointestinal flora and in the treatment of gastrointestinal symptoms. The present invention relates to stabilization of any viable bacteria in a composition for application. The bacteria are preferably selected among the genera Lactobacillus, Leuconostoc, Bifidobacterium, Pediococcus, Lactococcus, Streptococcus Aerococcus, Carnobacterium, Enterococcus, Oenococcus, Sporolactobacillus, Tetragenococcus, Vagococcus, and Weissella.

The preferred microorganisms are in particular bacteria. The probiotic bacteria is preferably selected from the group comprising Lactococcus lactis, Lactobacillus rhamnosus, Lactobacillus plantarum, Lactobacillus helveticus, Lactobacillus jensenii, Lactobacillus acidophilus, Lactobacillus bulgaricus, Lactobacillus amylovorus, Lactobacillus amylolyticus, Lactobacillus alimentarius, Lactobacillus aviaries, Lactobacillus delbrueckii, Lactobacillus diolivorans, Lactobacillus farciminis, Lactobacillus gallinarum, Lactobacillus casei, Lactobacillus crispatus, Lactobacillus gasseri, Lactobacillus johnsonii, Lactobacillus hilgardii, Lactobacillus kefiranofaciens, Lactobacillus kefiri, Lactobacillus mucosae, Lactobacillus panis, Lactobacillus paraplantarum, Lactobacillus pontis, Lactobacillus sakei, Lactobacillus saliverius, Lactobacillus sanfraciscensis, Lactobacillus paracasei, Lactobacillus pentosus, Lactobacillus cellobiosus, Lactobacillus collinoides, Lactobacillus coryniformis, Lactobacillus crispatus, Lactobacillus curvatus, Lactobacillus brevis, Lactobacillus buchneri, Lactobacillus fructivorans, Lactobacillus hilgardii, Lactobacillus fermentum, Lactobacillus reuteri, Lactobacillus ingluviei, Weissella viridescens, Bifidobacterium bifidum, Bifidobacterium adolescentis, Bifidobacterium breve, Bifidobacterium longum, Bifidobacterium animalis, Carnobacterium divergens, Corynebacterium glutamicum, Leuconostoc citreum, Leuconostoc lactis, Leuconostoc mesenteroides, Leuconostoc pseudomesenteroides, Oenococcus oeni, Pasteuria nishizawae, Pediococcus acidilactici, Pediococcus dextrinicus, Pediococcus parvulus, Pediococcus pentosaceus, Probionibacterium freudenreichii, Probionibacterium acidipropoinici, Enterococcus faecium, Enterococcus faecalis, Streptococcus thermophilus, Bacillus amyloliquefaciens, Bacillus atrophaeus, Bacillus dausii, Bacillus coagulans, Bacillus flexus, Bacillus fusiformis, Bacillus lentus, Bacillus licheniformis, Bacillus mega-terium, Bacillus mojavensis, Bacillus pumilus, Bacillus smithii, Bacillus subtilis, Bacillus vallismortis, Geobacillus stearother-mophilus or mutants thereof.

In another aspect of the invention the probiotic microorganism is selected from the genera related to the natural healthy skin microbiome including genera Probionibacterium, Cutibacterium, Staphylococcus, Corynebacterium, Malassezia, Aspergillus, Cryptococcus, Rhodotorula, and/or Epicoccum.

In a preferred embodiment of the invention the probiotic strain is Staphylococcus epidermidis, Staphylococcus hominis, Cutibacterium acnes (Probionibacterium acnes) or any combinations thereof.

In a preferred embodiment of the invention the probiotic strain is a Gram-positive bacteria. In one preferred embodiment of the invention the composition comprises at least one strain selected from the group consisting of Lactobacillus plantarum LB356R (DSM 33094), Weissella viridescens LB10G (DSM 32906), Lactobacillus plantarum LB113R (DSM 32907), Lactobacillus plantarum LB244R (DSM 32996), Lactobacillus paracasei LB116R (DSM 32908), Lactobacillus paracasei LB28R (DSM 32994), Lactobacillus brevis LB152G (DSM 32995) and Leuconostoc mesenteroides LB276R (DSM 32997) or mutant strains.

In a preferred embodiment the oil gel embedded microorganism is selected from the list but not restricted to: Bifidobacterium lactis DSM10140, B. lactis LKM512, B. lactis DSM 20451, Bifidobacterium bifidum BB-225, Bifidobacterium adolescentis BB-102, Bifidobacterium breve BB-308, Bifidobacterium longum BB-536 from Zaidanhojin Nihon Bifizusukin Senta (Japan Bifidus Bacteria Center), Bifidobacterium NCIMB 41675 described in EP2823822. Bifidobacterium bifidum BB-225, Bifidobacterium adolescentis BB-102, Bifidobacterium breve BB-308, Bifidobacterium lactis HNO19 (Howaru) available from DuPont Nutrition Biosciences ApS, Bifidobacterium lactis DN 173 010 available from Groupe Danone, Bifidobacterium lactis Bb-12 available from Chr. Hansen A/S, Bifidobacterium lactis 420 available from DuPont Nutrition Biosciences ApS, Bifidobacterium breve Bb-03, B. lactis BI-04, B. lactis Bi-07 available from DuPont Nutrition Biosciences ApS, Bifidobacterium bifidum Bb-02, Bifidobacterium bifidum Bb-06, Bifidobacterium longum KC-1 and Bifidobacterium longum 913 (DuPont Nutrition Biosciences ApS), Bifidobacterium breve M-16V (Morinaga) and/or a Lactobacillus having a probiotic effect and may be any of the following strains; Lactobacillus rhamnosus LGG (Chr. Hansen), Lactobacillus acidophilus NCFM (DuPont Nutrition Biosciences ApS), Lactobacillus bulgaricus 1260 (DuPont Nutrition Biosciences ApS), Lactobacillus paracasei Lpc-37 (DuPont Nutrition Biosciences ApS), Lactobacillus rhamnosus HN001 (Howaru)available from DuPont Nutrition Biosciences ApS, Streptococcus thermophilus 715 and Streptococcus thermophilus ST21 available from DuPont Nutrition Biosciences ApS, Lactobacillus paracasei subsp. paracasei CRL431 (ATCC 55544), Lactobacillus paracasei strain F-19 from Medipharm, Inc. L. paracasei LAFTI L26 (DSM Food Specialties) and L. paracasei CRL 431 (Chr. Hansen), Lactobacillus acidophilus PTA-4797, L. salivarius Ls-33 and L. curvatus 853 (DuPont Nutrition Biosciences ApS). Lactobacillus casei ssp. rhamnosus LC705 is described in FI Patent 92498, Valio Oy, Lactobacillus DSM15527 (Bifodan), Lactobacillus DSM15526 (Bifodan), Lactobacillus rhamnosus GG (LGG) (ATCC 53103) is described in U.S. Pat. No. 5,032,399 and Lactobacillus rhamnosus LC705 (DSM 7061), Propionic acid bacterium eg. Propionibacterium freudenreichii ssp. shermanii PJS (DSM 7067) described in greater details in FI Patent 92498, Valio Oy, Nitrosomonas eutropha D23 (ABIome), Staphylococcus hominis strains A9, C2, AMT2, AMT3, AMT4-C2, AMT4-GI, and/or AMT4-D12. (all from Matrisys Bioscience), L. rhamnosus PB01, L. gasseri EB01, L. curvatus EB10, L. acidophilus 5, Bifidobacterium animalis ssplactis 12, Bifidobacterium longum 536 all available from Bifodan A/S. Staphylococcus epidermidis strains M034, M038, All, AMT1, AMT5-05, and/or AMT5-G6 (all from Matrisys Bioscience), L. plantarum YUN-V2.0 (BCCM LMG P-29456), L. pentosus YUN-V1.0 (BCCN LMG P-29455), L. rhamnosus YUN-S1.0 (BCCM LMG P-2961), Weissella viridescens LB10G (DSM 32906), Lactobacillus paracasei LB113R (DSM 32907), Lactobacillus plantarum LB244R (DSM 32996), Lactobacillus paracasei LB116R (DSM 32908), Lactobacillus brevis LB152G (DSM 32995), Lactobacillus paracasei LB28R (DSM 32994), Enterococcus faecium LB276R (DSM 32997), Leuconostoc mesenteriodes LB349R (DSM 33093), Lactobacillus plantarum LB316R (DSM 33091), Lactobacillus plantarum LB356R (DSM 33094), Lactobacillus plantarum LB312R (DSM 33098); and/or any combinations hereof.

The use of viable probiotics for topical application is very limited and most products are based on lysates of the in-activated probiotic strain to overcome the problems of maintaining viability of the microorganisms in the topical composition. The problems observed when formulating live probiotic strains in gels, serums, emulsions, lotions and the like for topical application on the skin of mammals are lack of viability and stability.

Compositions for topical applications are typically to be stable for months at room temperature, this is a major problem for maintaining viability of live probiotic microorganisms in skin care products.

Another problem is activation of the probiotic strain when applied on the skin of a mammal. If the probiotic strain is microencapsulated following the procedures used for stabilization of probiotics for oral consumption then the microcapsules are designed to protect the live probiotic strain in the gastrointestinal fluids and will thus not dissolve on the skin surface. Therefore, the probiotic strain will not be released from the encapsulation and thereby not able to establish a binding, a metabolism or colonization of the probiotic strain on the skin surface or on mucous membranes.

The present invention solves the problem of stabilization of the live probiotic strain in an oil gel for topical use on skin or mucous membranes.

It was completely surprising that embedding the microorganisms in an oil gel resulted in maintenance of viability and facilitated the probiotic effect on the skin or mucous membranes.

It will be understood that in the following, preferred embodiments referred to in relation to one broad aspect of the invention are equally applicable to each of the other broad aspects of the present invention described above. It will be further understood that, unless the context dictates otherwise, the preferred embodiments described below may be combined.

When used herein, the term topical includes references to formulations that are adapted for application to body surfaces (e.g. the skin or mucous membranes). Mucous membranes that may be mentioned in this respect include the mucosa of the vagina, the penis, the urethra, the bladder, the anus, the nose and the ear.

In a preferred embodiment the oil gel is formulated for vaginal application.

In a preferred embodiment the oil gel is formulated for nasal application.

The present invention discloses new compositions and methodologies for stabilization of live probiotic strains in a composition for topical use to mucous membranes.

The utilization of these compositions comprising probiotic bacteria further facilitate the probiotic effects on skin of both humans and animals.

The present invention discloses methodologies for the formulation of oil gels comprising viable microorganisms.

The present invention further provides a therapeutic composition for the treatment or prevention of an skin disorder, comprising a therapeutically-effective concentration of one or more live species or strains or live biotherapeutic products within a pharmaceutically-acceptable carrier suitable for topical administration on the skin or mucous membranes of a mammal, wherein said probiotic strain possesses the ability to maintain viable in the composition at room temperature and be released when applied to the skin surface.

In another aspect, the invention relates to a composition comprising a pharmaceutically or cosmetically acceptable vehicle or excipient. It is preferable for the composition to be present in solid, liquid, or viscous form.

The composition is preferably in the form of a gel. More preferable the composition is an oil gel.

The composition is preferably in the form of a gel comprising less than 10% water, more preferable the composition is an oil gel comprising less than 5% water, more preferable the composition is an oil gel comprising less than 1% water, more preferable the composition is an oil gel comprising less than 0.5% water, more preferable the composition is an oil gel comprising less than 0.1% water, more preferable the composition is an oil gel comprising less than 0.05% water.

In one preferred embodiment the invention relates to a topical composition for skin of either humans or animals.

In a further preferred embodiment the oil gel composition comprises at least one carbonhydrate, and at least one of fat embedded microorganism.

The composition may advantageously further comprise other probiotics, prebiotics, or other active substances and/or may preferably also contain one or more of the following substances selected from antioxidants, vitamins, coenzymes, fatty acids, amino acids and cofactors.

In a preferred embodiment of the invention, the composition is a topical pharmaceutical, veterinary, cosmetic, vaginal care or skin care product.

The composition according to the present invention may be suitable for the prophylaxis or treatment of a disease, dysfunction or disorder of a mucous membrane.

In an embodiment of the present invention the mucous membrane may be the vagina, the penis, the urethra, the bladder, the anus, the nose and the ear.

The composition according to the present invention may be suitable for the treatment or prevention of a skin disease, preferably the skin disease is selected from eczema, dermatitis, atopic dermatitis, carbuncle, cellulitis, rosacea, psoriasis, diaper rash, impetigo, psoriasis, acne and wounds.

The composition preferable contains one or more prebiotic sources for the probiotic strain to restore metabolism on the skin or mucous membrane.

In a preferred embodiment of the invention the composition comprising at least one live probiotic strain for use in the treatment of a skin or mucous membrane disorder or dysfunction.

As used herein, and as well-understood in the art, “treatment” is an approach for obtaining beneficial or desired results, including clinical results. For purposes of this subject matter, beneficial or desired clinical results include, but are not limited to, alleviation or amelioration of one or more symptoms, diminishment of extent of disease, stabilized (i.e., not worsening) state of disease, prevention of disease, delay or slowing of disease progression, and/or amelioration or palliation of the disease state. The decrease can be a 10 percent, 20 percent, 30 per-cent, 40 percent, 50 percent, 60 percent, 70 percent, 80 percent, 90 percent, 95 percent, 98 percent or 99 percent decrease in severity of complications or symptoms.

In addition, the invention relates to compositions containing these oil gel embedded microorganisms, in particular for use in treating skin or mucous membrane disorders or skin or mucous membrane diseases or skin or mucous membrane microbiota dysfunctions, in products for topical use.

In preferred embodiments the oil gel embedded microorganisms are used for treatment of a disease is selected from the group of skin diseases comprising psoriasis, atopic dermatitis, dry skin, sensitive skin, acne prone skin, acne, hyperpigmented skin, aged skin, allergy, eczema, rashes, UV-irritated skin, photodamaged skin, detergent irritated skin (including irritation caused by enzymes used in washing detergents and sodium lauryl sulphate), Rosacea, thinning skin (e.g. skin from the elderly and children), bacterial vaginosis, urinary tract infections.

In a preferred embodiment the composition is used for vaginal care.

In one preferred embodiment of the invention, the composition comprising at least one oil gel embedded probiotic microorganism according to the invention is used on the skin of patients with inflammatory skin diseases.

In a preferred embodiment of the invention the skin disorder is associated with atopic dermatitis, eczema, impetigo, acne, burns, diaper rash, wounds.

The composition of the invention may be used curatively or prophylactically, for example, as a probiotic treatment of the skin or mucous membranes.

In one preferred embodiment of the invention, the composition comprising at least one oil gel embedded probiotic microorganism according to the invention is used on the vagina mucous membrane.

Vegetable oils contains natural antioxidants, in a preferred embodiment of the invention further antioxidants are incorporated into the composition. Antioxidants are preferred Vitamin E (0.25 to 10 wt %) and/or Rosemary extract (0.1 to 0.75 wt %).

A “decrease” in viability may be “statistically significant” as compared to the viability determined at the time of formulating the composition. Decrease is measured as a log reduction and may include a log reduction of 0.1, 0.5, 1, 1.5, 2, 2.5, 3, 3.5, 4, 4.5 or 5. “Viability” of microorganisms is measured as Colony Forming Units CFU/ml. A “decrease” in viability of microorganisms may be determined as the difference in CFU/ml as compared to the CFU/ml at the time of formulating the composition.

The microorganisms according to the invention are preferably in isolated or purified form, where the term “isolated” means in particular that the microorganism is cultivated as a monoculture and is derived from the culture medium including their natural medium, for example. The term “purified” is not restricted to absolute purity.

The microorganisms may advantageously be present in viable spray-dried and/or lyophilized form.

In a preferred embodiment of the invention the probiotic strain is used as a live isolated microorganism in a dried form. Suitable methods for cryoprotection are known to those skilled in the art and includes freeze drying or lyophilization involving different cryoprotectants.

Freeze drying, also known as lyophilization or cryodesiccation is a low temperature dehydration process which typically involves freezing the product, lowering pressure, then removing the ice by sublimation. Lyophilization of microorganisms maintain viability of the microorganism.

In a preferred embodiment of the invention the strain is used as a viable isolated strain.

In a preferred embodiment of the invention the strain is used as a viable isolated lyophilized strain.

In addition, it is preferable for the microorganism to be present in the composition in an amount by weight of 0.001 wt % to 20 wt %, preferably 0.005 wt % to 10 wt %, especially preferably 0.01 wt % to 5 wt %. A preferred embodiment of the present invention involves the administration of from approximately 1×10³ to 1×10¹⁴ CFU of viable bacteria per gram of the composition, more preferably from approximately 1×10⁴ to 1×10¹⁰, and most preferably from approximately 1×10⁵ to 1×10⁹ CFU of viable bacteria per gram of composition.

In one preferred embodiment of the invention the dosage of live probiotic microorganisms in the composition is above approximately 1×10⁴ CFU of viable bacteria per gram of the composition, preferably above approximately 1×10⁵.

The viable, lyophilized microorganisms are embedded into the oil gel in lumps with more than 10 viable cells per lump.

Lumps in the oil are less than 100 μm in diameter, typically in the interval from 5 μm to 95 μm in diameter.

Preferable the lumps have a diameter from 10 μm to 90 μm.

Lumps can form clusters in the oil gel. The clusters comprises more than one lump, each lump with a diameter of approximately 5 μm to 100 μm

Where the condition to be treated involves a live biotherapeutic product (probiotic microorganism) with a therapeutic effect on a disorder, the concentration of viable microorganism in the composition is at the concentration needed for obtaining the therapeutic effect of the probiotic microorganism.

Another surprising advantage of the preferred composition is that the microorganisms are able to activate on the skin and re-establish metabolic activity.

It will be clear to those skilled in the art that here, as well as in all the statements of range given in the present invention, characterized by such terms as “about” or “approximately,” that the precise numerical range need not be indicated with expressions such as “about” or “approx.” or “approximately,” but instead even minor deviations up or down with regard to the number indicated are still within the scope of the present invention.

A “mammal” include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; piglets; sows; poultry; turkeys; broilers; minks; goats; cattle; horses; and non-human primates such as apes and monkeys.

Preferable the composition is for topical use on human skin or human mucous membranes. An “effective amount” depends upon the context in which it is being applied. In the context of administering a composition comprising a viable microorganism topically on a skin or mucous membrane surface, an effective amount will be the number of viable microorganisms determined as CFU/gram which has a probiotic effect on skin or mucous membranes.

In one aspect of the invention the composition comprising the microorganism and a prebiotic. “Prebiotics” are components that increase the growth of specific microorganisms. “Synbiotics” are compositions comprising at least one probiotic and at least one prebiotic.

Such compositions are understood to encourage the growth of beneficial microorganisms (e.g. the probiotic). Thus, powerful synbiotics are based on a combination of specific strains of probiotic microorganisms with carefully selected prebiotics. They can lead to an important health benefit to a mammal.

According to another aspect of the present invention there is provided a probiotic composition comprising the probiotic micro-organism and at least one more active ingredient.

Prebiotics refer to chemical products that induce the growth and/or activity of commensal microorganisms of the microbiota (e.g., bacteria and fungi) that contribute to the well-being of their host. Prebiotics stimulate the growth and/or activity of advantageous bacteria that colonize the skin.

Some oligosaccharides that are used as prebiotics are fructooligosaccharides (FOS), xylooligosaccharides (XOS), polydextrose, pectins, galactooligosaccharides (GOS) or human milk oligo saccharides (HMO). Moreover disaccharides like lactulose, lactose or some monosaccharides such as or tagatose can also be used as prebiotics.

The other active ingredient (or other ingredients) is not limited in any way. In a preferred aspect, at least one prebiotic compound is comprised in the composition of the invention, i.e. as other ingredient. In a very broad concept, prebiotics are all those compounds which can be metabolized by probiotics. Prebiotics can thus serve as a food source for probiotics. Prebiotics are well known in the art and when used in the present invention there is no particular limitation of the prebiotic as such. In preferred embodiments at least one prebiotic product in the composition is selected from the following compounds and compositions: carbohydrates, glucans, alpha-glucans, beta-glucans, mannan-oligosaccharides, inulin, oligofructose, human milk oligosaccharides (HMO), galactooligosaccharides (GOS), lactulose, lactosucrose, galactotriose, fructooligosaccaride (FOS), cellobiose, cellodextrins, cylodextrins, maltitol, lactitol, glycosilsucrose, betaine, Vitamin E or a variant thereof (wherein the variants are selected from alfa, beta, gamma, delta tocoferols, tocotrienols and tocomonoenols). Optionally, mannanoligosaccharides and/or inulin may be preferred.

HMOs include lacto-N-tetraose, lacto-N-fucopentaose, lacto-N-triose, 3′-sialyllactose, lacto-N-neofucopentaose, sialic acid, L-fucose, 2-fucosyllactose, 6′-sialyllactose, lacto-N-neotetraose and 3-fucosyllactose.

In a preferred embodiment at least one of the following prebiotic compounds are used in the topical composition of the invention; lactose, beta-glucans, mannan-oligosaccharides, inulin, oli-gofructose, galactooligosaccharides (GOS), lactulose, lactose, lactosucrose, galactotriose, fructo-oligosaccaride (FOS), cellobiose, cellodextrins, cylodextrins, maltitol, lactitol, glycosilsucrose, betaine, Vitamin E or a variant thereof (wherein the variants are selected from alfa, beta, gamma, delta tocoferols, tocotrienols and tocomonoenols), lacto-N-tetraose, lacto-N-fucopentaose, lac-to-N-triose, 3′-sialyllactose, lacto-N-neofucopentaose, sialic acid, 2-fucosyllactose, 6′-sialyllactose, lacto-N-neotetraose and 3-fucosyllactose. Optionally, lactose and/or mannan-oligosaccharides and/or inulin may be preferred.

Fucose, in particular L-fucose is believed to strengthen natural defense of skin, stimulate epidermis immune defense and/or pre-vent and/or treat cutaneous autoimmune disease. In one preferred embodiment of the invention the composition comprises L-fucose and/or D-fucose.

In one preferred embodiment of the invention the composition further comprises L-fucose and/or D-fucose in a concentration in the composition of 10 mM to 500 mM.

According to still further features in the described preferred embodiments the composition comprising the microorganism of the invention further comprises at least one further probiotic microorganism selected from the group consisting of bacteria, archaea, phages, virus, yeasts or molds.

In a preferred embodiment the at least one further probiotic microorganism is a bacteria.

In one embodiment of the invention the oil gel is used as an hydrophobic phase in an emulsion. An emulsion is a mixture of two or more liquids that are normally immiscible (i.e.: oil and water). Emulsions are part of a more general class of two-phase systems of matter called colloids. Although the terms colloid and emulsion are sometimes used interchangeably, emulsion is used when both the dispersed and the continuous phase are liquid. In an emulsion, one liquid (the dispersed phase) is dispersed in the other (the continuous phase).

In a preferred embodiment of the invention, the fat embedded microorganism is suspended in an oil gel and further incorporated into an emulsion comprising a water phase and an optional a second oil or fat phase, wherein the oil gel phase comprises the microorganisms embedded in gelled oil.

An “oil” of the invention is an oil being liquid at storage temperature, thus the liquid oil has a freezing point below 25 degrees celsius. More preferable the freezing point is below 5 degrees Celsius, and even more preferably the freezing point is below 0 degrees Celsius.

The oil of the invention is not a solidified oil (butter or fat). Butters and fats are according to the invention not considered as oils suitable for oil gels. Butters need to be liquid fractionated to oils before gelling. E.g. coconut, cacao or shea butters.

In a preferred embodiment the oil is a vegetable oil which can be absorbed by the skin or the mucous membrane.

In a preferred embodiment of the invention the oil is a vegetable oil selected from almond oil, sunflower oil, hemp oil, CBD oil, cannabis oil, Evening prim rose, Borage oil, acai oil, Almond sweet oil, Rose Hip oil, jojoba oil, Jojoba Golden oil, Camomile oil, Calendula oil, Sea buck-thorn oil, Jafflower oil, castor oil, olive oil, linseed oil, apricot kernel oil, argan oil, camelina oil, comfrey oil, grape seed oil, kiwi seed oil, mullein oil, peach kernel oil, thistle oil and sesame oil.

In one preferred embodiment of the invention, the composition comprising at least one oil gel embedded probiotic microorganism, wherein the oil is selected from sunflower oil, jojoba oil and almond oil.

The vegetal oil may comprise at least one of: acai, acai berry, almond sweet, aloes vera, andiroba, apricot kernel, arnica, argan, avocado, babassu, boabab, black berry seed, black cumin, black currant seed, blueberry, borage, brazil nut, brocoli seed, buriti, calendula, camellia seed, cannabis oil including CBD and THC, canola, copaiba balsam, cape chestnut (yangu), carrot (daucus carrota), castor, chardonnay grape, chaulmoogra, cherry Kernel, chia seed, chickweed, coconut, coconut fractionated, cotton seed, comfrey, corn, crambe seed, cranberry seed, cucumber seed, echium seed, evening primrose, emu, flax seed, grape seed, hazelnut, hemp seed, horsechest nut seed, jojoba, karanj seed, kiwi seed, kukuinut, macadamia nut, marula, marshmallow, manketti, meadowfoam, milk thistle seed, moringa, mullein, mustard seed, neem, olive, palm, papaya seed, passionflower seed, peach kernel, peanut, perilla, pomegranate, Pentaclethra macroloba, pumpkin seed, raspberry seed, rice bran, rosehip, St. John's Wort oil, safflower, sea buckthorn pulp, sheabutter oil, sesame roast-ed, sesame seed, soya been, sunflower, tamanu (Calophyllum In-ophyllum), thistle, tomato, turkey red, sangre de drago, walnut, watermelon seed, wheatgerm, Abyssinian, Colza, bees wax, lanolin, linseed, mortierella oil, ongokea, paraffinum liquid, peacan, Pegui, Poppy seed, Pracaxi, rapeseed, soybean, tall, tung, veronica, Wheat germ, yangu seed and any combination thereof.

In a preferred embodiment of the invention the oil gel composition is used as a topical composition with essential no water in the composition.

In a preferred embodiment of the invention the oil gel composition with the oil gel stabilized microorganisms are used as ingredient in a further formulation of the oil gel embedded microorganisms.

In a preferred embodiment the composition of the further formulation is an emulsion consisting of a hydrophilic phase and a hydrophobic phase wherein the hydrophobic phase comprises oil gel embedded viable microorganisms.

According to still further features in the described preferred embodiments the probiotic microorganisms is capable of proliferating and colonizing on and/or in the mammalian skin or mucous membranes.

The present invention successfully addresses the shortcomings of the presently known compositions for topical use. Known compositions for topical use are either not able to maintain the viability of the microorganisms or the microorganisms are not able to activate on the skin surface.

The present invention provides several advantages. In particular, viability of the microorganisms is kept in the composition even at storage at room temperature. The microorganisms activated by the temperature and moisture of the skin releasing the microorganisms from the oil gel as the oil is absorbed by the skin.

In a further aspect, this invention provides methods for preparing a topical composition comprising a oil gel embedded viable microorganism.

In a preferred variation, the microorganism is a lyophilized culture. Also, preferably the oil gel is low in free moisture (i.e., Aw less than 0.4) so as to minimize exposure of the dried viable microorganism to moisture and to avoid activation of the microorganism.

The oil composition comprising the oil gel embedded microorganisms can be further processed.

The method can further involve the following step. The oil composition comprising oil gel embedded microorganisms can be admixed with a hydrophilic composition allowing for emulsification, optionally along with any supplemental soluble ingredients. The oil gel embedded microorganisms will stay in the oil gel. The oil gel can be either the continuously phase or the dis-continuously phase of the emulsion. Preferably, the oil gel may be the continuously phase.

Provided is also a procedure to produce a composition comprising an oil embedded microorganism for topical use.

The inventors of the present invention surprisingly found that providing the oil gel according to the present invention it was possible to maintain a significant improvement in the viability of the embedded microorganism. The improved viability may be provided for more than 1 month, such as for at least 2 months, e.g. for at least 4 months, such as for at least 6 months, e.g. for at least 8 months, such as for at least 12 months, e.g. for at least 1½ year, such as for at least 2 years.

Preferably, the maintained viability may relate to at least 50% of the microorganisms are viable relative to the amount of microorganisms originally added to the oil gel composition; such as at least 75%; e.g. at least 85%; such as at least 90%; e.g. at least 95%.

In a preferred embodiment of the present invention the method for providing an oil gel composition according to the present invention may comprise the following steps;
a. Lyophilization of a viable microorganism resulting in a lyophilized biomass of at least 10² CFU/g biomass;
b. Embedding the lyophilized biomass in a mixture of an oil and an organogelator;
c. Immobilizing the lyophilized biomass in a three-dimensional network formed by the organogelator.

In an embodiment of the present invention the three-dimensional network may be formed by stirring the oil, the organogelator (the oil based viscosity increasing agent) and the viable microorganism. Preferably, stirring may be performed in the range of 100-800 rpm; such as in the range of 300-650 rpm; e.g. in the range of 450-550 rpm; such as about 510 rpm.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

### Example 1

Cold pressed organic Jojoba oil (Simmondsia chinensis seed oil) was obtained from Hedenhus, Denmark. Organic refined sweet almond oil was obtained from Nardos Cosmeceuticals and organic sunflower oil (Helianthus annuus seed oil) was obtained from Hedenhus, Denmark.

Oilkemia 5S polymer was obtained from Lubrizol.

Lyophilized Lactobacillus rhamnosus LGG was obtained from the commercially available product Culturelle Probiotics Pro-Well, lot 18024CGM15 (DSM). Capsules for oral consumption were broken and the lyophilized viable L. rhamnosus LGG strain were used in the oil gels as a both lyophilized powder as well as the strains were grown on MRS agar plates over night for 24 hours at 37 degrees Celsius and used in the oil as a fresh cultured viable strain.

Lactobacillus plantarum LB244R (DSM 32996) were grown in MRS broth over night for 24 hours at 37 degrees Celsius and harvested by centrifugation, the cells were lyophilized over night using either skim-milk powder or sorbitol as cryoprotectant.

Oil gel were produced using the following procedure:

Step 1 Each oil was heated with 1% (w/w) Oilkemia 5S polymer until the polymer is dissolved (approximately 85 degrees Celsius)

Step 2 Oils were cooled to below 30 degrees while stirring

Step 3 viable probiotic strain was mixed into the oil either as a lyophilized powder or as a colony from an agar plate with a fresh overnight culture.

Colony forming unit (CFU) was determined for each oil. The oils were allowed to stand (no stirring) and an oil gel comprising viable probiotic strains were generated. The oil gels were stored for stability testing at the following temperatures: 20, 25 and 37 degrees Celsius.

TABLE 1
Stability determined as viable strain at time = 0, and 1, 4, 8 and 12
weeks respectively. Shown for storage temperature 20 degrees Celsius. Cell
counts are measured as CFU/g of oil gel. (Average of a triplet CFU determination)

| | | T = 1 | T = 4 | T = 8 | T = 12 |
|---|---|---|---|---|---|
| Oil gel | T = 0 | week | weeks | weeks | weeks |
| Jojoba w. | 7.4 × 10⁸ | 6.1 × 10⁸ | 2.1 × 10⁸ | 4.3 × 10⁷ | 1.0 × 10⁷ |
| Lyophilized LGG | | | | | |
| Jojoba w. | 8.0 × 10⁹ | 4.4 × 10⁶ | 1.7 × 10³ | 5.4 × 10² | 3 |
| Fresh cultured LGG | | | | | |
| Jojoba w. Lyophilized | 9.9 × 10⁷ | 9.8 × 10⁶ | 8.4 × 10⁶ | 4.9 × 10⁶ | 5.4 × 10⁶ |
| LB244R_Skimmilk | | | | | |
| Jojoba w. Lyophilized | 3.4 × 10⁷ | 4.7 × 10⁷ | 8.2 × 10⁷ | 7.3 × 10⁶ | 9.6 × 10⁶ |
| LB244R_Sorbitol | | | | | |
| Almond oil w. | 5.5 × 10⁸ | 4.0 × 10⁸ | 4.8 × 10⁸ | 1.4 × 10⁸ | 8.7 × 10⁷ |
| Lyophilized LGG | | | | | |
| Almond oil w. | 3.7 × 10¹¹ | 5.4 × 10⁶ | 3.9 × 10³ | 7.2 × 10¹ | 17 |
| Fresh cultured LGG | | | | | |
| Almond oil w. | 8.2 × 10⁷ | 2.1 × 10⁷ | 9.0 × 10⁷ | 2.2 × 10⁶ | 4.6 × 10⁶ |
| Lyophilized | | | | | |
| LB244R_Skimmilk | | | | | |
| Almond oil w. | 6.5 × 10⁷ | 7.8 × 10⁷ | 6.3 × 10⁷ | 5.3 × 10⁷ | 8.4 × 10⁶ |
| Lyophilized | | | | | |
| LB244R_Sorbitol | | | | | |
| Sunflower oil w. | 2.4 × 10⁸ | 2.7 × 10⁸ | 1.1 × 10⁸ | 5.3 × 10⁸ | 1.9 × 10⁸ |
| Lyophilized LGG | | | | | |
| Sunflower oil w. | 6.9 × 10¹⁰ | 1.4 × 10⁵ | 6.5 × 10² | 621 | 2 |
| Fresh cultured LGG | | | | | |
| Sunflower oil w. | 9.2 × 10⁷ | 8.0 × 10⁷ | 1.3 × 10⁸ | 2.5 × 10⁷ | 9.4 × 10⁷ |
| Lyophilized | | | | | |
| LB244R_Skimmilk | | | | | |
| Sunflower oil w. | 8.7 × 10⁷ | 9.7 × 10⁷ | 1.0 × 10⁷ | 6.7 × 10⁷ | 7.2 × 10⁶ |
| Lyophilized | | | | | |
| LB244R_Sorbitol | | | | | |

Control oils with no polymer was included for all strains. Both fresh and lyophilized strains sediment in oils which are not gelled, for all strains a lower CFU was determined in the oil as compared to the oil gel. The fresh cultured cells were only viable for 4 weeks in the 3 different oils when the oils are not gelled. For the lyophilized oils a log reduction of 1-2 log was observed in the control oils without polymer.

All three oils formed a semi-solid gel while using Oilkemia 5S polymer at 1% w/w.

### Example 2

Oils used for gelling:

Sample 1: Almond oil

Sample 2: Borage oil

Sample 3: Almond sweet oil

Sample 4: Rose Hip oil

Sample 5: Jojoba Golden oil

Sample 6: Camomile oil

Sample 7: Calendula oil

Sample 8: Sea buckthorn oil

Sample 9: Jafflower Evening prim rose oil

Sample 10: Sesame oil

Oilkemia 5S polymer was obtained from Lubrizol

EstoGel M was obtained from PolymerExpert

Oil gels were produced following the procedure:

Step 1: heating the oil with one of the polymers until solubilization

Step 2: stirring the oil-polymer mixture while cooling to room temperature

Step 3: adding lyophilized LB244R.

The two polymers were used in the concentrations: 0.1%, 1% and 5% w/w.

The oil gels have different viscosity depending on oil and the concentration of polymer, most of the oil gels with 5% w/w polymers are solid or partly solid oil gels.

The viability of lyophilized LB244R in the oil gels were determined after 2 weeks and for all gels the viability was unchanged after 2 weeks in the oil gels and better viability was obtained in the oil gels as compared to the oils.

### Example 3

Example 2 were performed using same procedure for making an oil gel. In this experiment two strains Leuconostoc mesenteroides LB349R (DSM 33093) and Weissella viridens LB10G (DSM32906) were grown in MRS broth over night for 24 hours at 37 degrees Celsius and harvested by centrifugation, the cells were lyophilized overnight.

For both strains a significant stability was obtained, thus 100% viability was maintained after 2 weeks storage of the oil gels at 37 degrees Celsius.

### Example 4

Oils used for gelling:

Jojoba oil (Natura-Tec)

Almond oil refined organic (Gustavheess)

Sunflower oil (Bressmer & Francke)

Mix of jojoba sunflower oil 1:1

Mix of jojoba almond oil 1:1

Wax for gelling:

Sunflower wax (Kahlwax 6607H)

Mix vegetable wax, Phytowax (Kahlwax 2225)

Rapeseed wax (Kahlwax 6237)

Olive oil wax (Natura-Tec OC wax)

Microorganisms:

Lactobacillus plantarum LB244R (DSM 32996)

Lyophilized Lactobacillus plantarum LB244R (DSM 32996)

Lyophilized Lactobacillus plantarum LB356R (DSM 33094)

Oil gels were prepared according to the following procedures:

Waxes are melted and mixed with the oils, using 0.5, 1, 5, 10, 15, 20, 25 or 30% (w/w) wax. Gentle stirred while cooling down to 25 degrees Celsius. Bacterial cells were prepared as described in example 1 and added to a concentration of approximately 10⁸ CFU/g of gel, gels were allowed to stabilize in structure for 24 hours at room temperature before stability storage.

Oil gels were produced for each oil-wax-bacteria combination and stored at 5, 25 and 37 degrees Celsius. Viability of the strains was determined in each gel after 24 hours and thereafter every second week.

For all combinations of oil, wax and bacteria, a solid gel was obtained when using 25 and 30% oil wax for gelling. For 0.5 and 1% of oil wax all combinations resulted in gelled oils still being liquid.

Lyophilized cells were significantly more stable in the gels than non-lyophilized cells, and maintained viability for +12 months, whereas the non-lyophilized cells declined in viability already after 2 weeks and were dead after 10 weeks. No significant difference, between the 3 oils or 2 mixtures of oils, were observed.

The most significant parameter for viability was the water content in the gel. Removing water by lyophilization of the cells before creating the gel had influence on viability.

For plate counting 5% polysorbate was used in the dilution buffer (PBS) as detergent to extract cells from the oil gel. The fluent gel was centrifuged to pellet the microorganisms, the fluent gel was removed from pellet and pellet was resuspended in PBS and diluted for plate counting.

### Example 5

Distribution and size of the embedded microorganism was determined for the compositions in example 4 by contrast phase microscopy and image analysis using the oCelluScope (BioSense Solution, Denmark).

For stability and dispersion into the oil it was measured that dispersion of lumps of lyophilized microorganisms in the size of 5-100 μm in diameter created the best distribution in the oil gel and resulted in improved viability of the microorganisms.

Size of the lumps can be controlled by the speed of mixing and depends on the mixing equipment and the viscosity of the oil gel at the temperature of mixing.

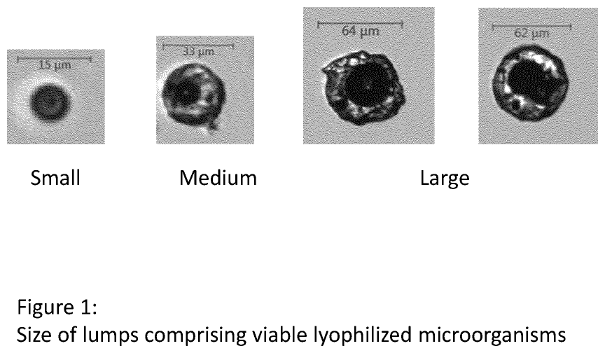

Oil gels from example 4 were analysed, example of image in FIG. 1.

FIG. 1 shows the size range obtained of the oil lumps. The lumps shown are in an oil gel comprising 1:1 jojoba and almond oil, 20% olive oil wax and lyophilized L. plantarum LB244R (as described in example 4). A Velp Scientific MST digital magnetic stirrer was used at 510 rpm to generate the lumps in a size of 5-100 μm in diameter.

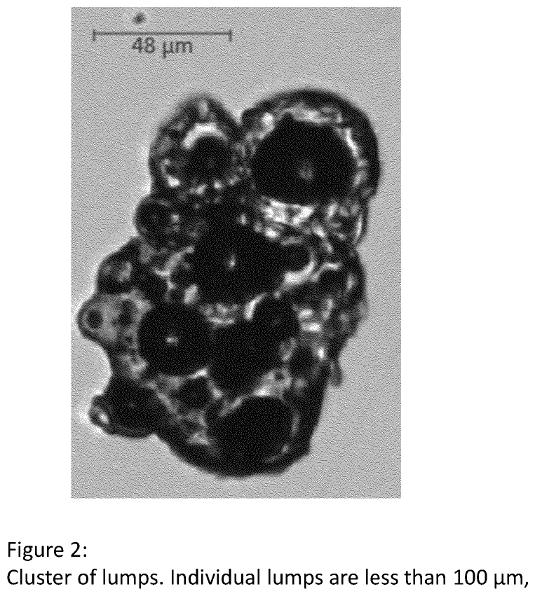

All sizes of lumps in all the compositions tested in example 4 had a size of the individual lump below 100 μm. All larger clumps of microorganisms observed in the oils were all related to the lumps forming clusters of individual lumps (FIG. 2), wherein each lump had a size less than 100 μm. Clustering of the lumps did not affect the viability of the microorganisms in the lump.

Viability of the microorganisms in the lumps were determined by image analysis in the oCelluScope. Oil gel is smeared in a thin layer in 6 well microtiter plates 10-20 μm thick and a thin layer of liquid MRS medium was added on top of the gel, out growth from the lumps was followed by image analysis.

### REFERENCES

Favaro-Trindade et al., (2011) CAB Reviews: Perspectives in Agriculture, Veterinary Science, Nutrition and Natural Resources 6:1-8
WO18185432

1. A composition comprising an oil gel comprising an oil, an oil based viscosity increasing agent and at least one viable microorganism, wherein the oil based viscosity increasing agent is an organogelator and wherein the organogelator is selected from plant wax, vegetable oil wax, hydrogenated vegetable wax and polyurethane polymers; and/or wherein the at least one viable microorganism is a lyophilized microorganism distributed in the composition in lumps of a size less than 100 μm.

2. (canceled)

3. The composition according to claim 1, wherein the oil has a melting point below 20 degrees Celsius.

4. The composition according to claim 1, wherein the oil has a melting point below 15 degrees Celsius.

5. The composition according to claim 1, wherein the composition is an oil gel at 20 degrees Celsius.

6. The composition according to claim 1, wherein the oil is a vegetable oil.

7. The composition according to claim 1, wherein the viable microorganism is a lyophilized microorganism.

8. (canceled)

9. The composition according to claim 6, wherein the lyophilized microorganism is a lactic acid bacterium.

10. (canceled)

11. The composition according to claim 1, for treatment or prevention of a disorder or disease.

12. The composition according to claim 1, for the prophylaxis or treatment of a disease, dysfunction or disorder of a mucous membrane.

13. The composition according to claim 9, wherein the mucous membrane is located on the vagina, the penis, the urethra, the bladder, the anus, the nose or the ear.

14. A method for the treatment or prevention of a skin disease or condition in a subject comprising administering to the subject a composition comprising an oil gel comprising an oil, an oil based viscosity increasing agent and at least one viable microorganism.

15. (canceled)

16. The method of claim 14, wherein the skin disease or condition is selected from eczema, dermatitis, atopic dermatitis, carbuncle, cellulitis, rosacea, psoriasis, diaper rash, impetigo, psoriasis, acne and wounds.

17. A method for providing an oil gel composition according to claim 1, the method comprising the following steps:
a. lyophilization of a viable microorganism resulting in a lyophilized biomass of at least 10² CFU/g biomass,
b. embedding the lyophilized biomass in a mixture of an oil and an organogelator, and
c. immobilizing the lyophilized biomass in a three-dimensional network formed by the organogelator,
wherein the oil based viscosity increasing agent is an organogelator and wherein the organogelator is selected from plant wax, vegetable oil wax, hydrogenated vegetable wax and polyurethane polymers; and/or wherein the lyophilized biomass is distributed in the composition in lumps of a size less than 100 μm.
